(19) United States
(12) Patent Application Publication
(10) Pub. No.: US 2008/0213850 A1
(43) Pub. Date: Sep. 4, 2008

(54) Pretreatment of Waste Mushroom Bed and Method of Converting the Same to Yield Sugars and Ethanol

(75) Inventors: Takafumi Shimoda, Niigata (JP); Kozo Nishibori, Niigata (JP); Takashi Shirouchi, Niigata (JP); Takayoshi Nakao, Niigata (JP); Yasuo Odaira, Niigata (JP); Yasushi Morikawa, Niigata (JP)

Correspondence Address: Davidson, Davidson & Kappel, LLC, 485 7th Avenue, 14th Floor, New York, NY, 10018, US

(73) Assignees: YUKIGUNI MAITAKE CO., LTD., Niigata (JP); NAGAOKA UNIVERSITY OF TECHNOLOGY, Niigata (JP)

(21) Appl. No.: 11/885,264

(22) PCT Filed: Feb. 21, 2006
(86) PCT No.: PCT/JP2006/303038

§ 371 Date: Aug. 27, 2007

(30) Foreign Application Priority Data
Feb. 28, 2005 (JP) 2005-053831

(51) Int. Cl. C12P 7/10; C07C 31/08

(52) U.S. Cl. 435/165; 568/700

(57) ABSTRACT

It is an objective of the present invention to develop a method of pretreating a waste mushroom bed so as to easily and efficiently obtain sugars and ethanol with the use of such waste mushroom bed. It is another objective of the present invention to develop a method of converting the pretreated waste mushroom bed to yield sugars and ethanol. According to the present invention, it has been found that the above objective can be achieved by maintaining a waste mushroom bed at 4° C. to 30° C. for 1 week or longer after harvesting of fruit bodies for conversion of the waste mushroom bed to yield sugars and ethanol.

### TECHNICAL FIELD

The present invention relates to waste mushroom bed pretreatment for conversion of woody biomass that can be used as an energy resource into sugars and ethanol, such woody biomass being contained in a waste mushroom bed that remains as a waste product after mushroom cultivation. The present invention also relates to a method of converting the pretreated waste mushroom bed to yield sugars and ethanol.

### BACKGROUND ART

At present, it is said that global reduction of carbon dioxide is necessary for prevention of global warming. Under such circumstances, the use of energy derived from unused biomass has been gaining attention. One reason for that is that such biomass can be so-called carbon-neutral biomass. Specifically, biomass contains carbon originally derived from atmospheric carbon dioxide that has been absorbed or fixed by plants. Thus, offsetting of carbon dioxide emissions (±0) resulting from energy extraction from such biomass is achieved by regenerating plants that can absorb emitted carbon dioxide. In addition, since it is possible to obtain fuel material such as ethanol or methane gas from biomass, biomass has been expected to replace fossil fuels that will be depleted in the future (Non-Patent Document 1).

It is difficult to convert biomass, especially woody biomass, into fuel material. This is because cellulose contained in such biomass, from which fuel material is obtained, is surrounded by persistent lignin, and thus it is difficult to use cellulose. Therefore, it is necessary to, for example, remove lignin and the like from cellulose contained in woody biomass so as to obtain cellulose in an available form (Non-Patent Documents 2, 3, and 4).

When conversion of woody biomass into sugars and ethanol is exclusively considered, there are roughly two types of methods for such conversion. One type of method is an acid hydrolysis method whereby cellulose in woody biomass is hydrolyzed to result in glucose with the use of acids and the like, following which glucose is converted into ethanol by fermentation. Such method has been examined and studied for years. However, reactions are carried out under strongly acidic, high-temperature, and high-pressure conditions, and thus costs of and maintenance costs for apparatuses that can be used under such conditions increase, which has been highly problematic (Non-Patent Documents 2 and 3).

Meanwhile, the other type of method is an enzymatic saccharification method whereby cellulose is degraded into glucose with the use of a cellulose-degrading enzyme (cellulase). Compared with the acid hydrolysis method, the enzymatic saccharification method is advantageous in terms of apparatus structure since reactions can be carried out under moderate conditions. In order to promote degradation, it is necessary for cellulase to come into contact with cellulose contained in a woody biomass. However, the presence of lignin as mentioned above and crystallization of cellulose prevent such contact. Thus, it is necessary to perform some sort of pretreatment prior to an enzymatic reaction. Examples of pretreatment for a method of enzymatically saccharifying a woody biomass include a variety of methods involving dilute sulfuric acid treatment, alkaline treatment, and fine pulverization. However, no definitive methods have been established (Non-Patent Documents 3 and 5).

Filamentous fungi have been known as organisms capable of degrading lignin in the natural world. Representative examples thereof include white-rot fungi. White-rot fungi release a powerful lignin-degrading enzyme for lignin degradation that gives decayed wood a whitish appearance. Most white-rot fungi belong to the Basidiomycetes, including fungi of many types of edible mushrooms such as “shiitake” (Lentinula edodes), “hiratake” (Pleurotus ostreatus), and “maitake” (Grifola frondosa) (Non-Patent Documents 6 and 7). White-rot fungi are used for treatment of woody biomass. For instance, a white-rot fungus having a capacity to degrade lignin, which is referred to as Ceriporiopsis subvermispora, is used in a wood chip pulping apparatus. Such treatment method is believed to allow cost-competitive paper production (Non-Patent Document 6).

Meanwhile, in recent years, maitake mushroom bed cultivation and the like is becoming common. This is because, in terms of artificial mushroom cultivation, large-scale mushroom bed cultivation with year-round air conditioning has become possible. A medium for mushroom bed cultivation is produced by mixing finely ground sawdust powder and mushroom nutrients, adequately adjusting the moisture content of the mixture, and packing the mixture in a bag or bottle. Such medium is sterilized and inoculated with mushroom hyphae, followed by culture for several months under adequate conditions. Then, after the spread of mushroom hyphae inside or outside the medium (which is referred to as a mushroom bed), mushroom fruit bodies are formed. In the natural world, the Basidiomycetes, to which mushroom fungi belong, must compete with other organisms. Consequently, the Basidiomycetes assimilate persistent wood. However, upon mushroom bed cultivation, such competition does not exist. Thus, in such case, the Basidiomycetes are believed to grow using non-wood nutrients that can be easily assimilated.

In practice, maitake is known to preferentially digest α-glucan (TFA-soluble glucan) derived from non-wood nutrients rather than β-glucan (cellulose) derived from wood (Non-Patent Document 8). Thus, it is assumed that unused cellulose, which is contained in sawdust powder, remains substantially intact in a mushroom bed (waste mushroom bed) that remains after harvesting of mushrooms produced by mushroom bed cultivation. Further, the weight content of sawdust powder (excluding moisture content) serving as a medium component is approximately 40% in the case of “bunashimeji” (Hypsizigus marmoreus) and 50% to 90% (mainly hardwood sawdust powder) in the cases of some types of mushrooms such as maitake, indicating that waste mushroom beds mainly consist of sawdust powder. Based on this, waste mushroom beds (especially waste mushroom beds used for cultivation of maitake or the like, which mainly comprise, as a medium component, sawdust powder) are expected to be used as woody biomass resources. Furthermore, large-scale cultivation of maitake and the like has been carried out in factories, and thus it is possible to obtain many waste mushroom beds simultaneously. However, at present, the use of waste mushroom beds is particularly limited to heat sources for boilers or the like.
Non-Patent Document 1: Kenji Yamaji (2002), Biomass Energy Characteristics and Energy Conversion/Utilization Technology (Biomass Energy no Tokusei to Energy Henkan/Riyo Gijutsu), NTS, pp. 3-36
Non-Patent Document 2: Shiro Saka et al. (2001), Biomass, Energy, and Environments, IPC, pp. 251-260
Non-Patent Document 3: Jun Sugiura (2002), Biomass Energy Characteristics and Energy Conversion/Utilization Technology (Biomass Energy no Tokusei to Energy Henkan/Riyo Gijutsu), NTS, pp. 283-312
Non-Patent Document 4: George P. Philippidis (1996), Handbook on Bioethanol, Taylor & Francis, pp. 253-285
Non-Patent Document 5: The-An Hsu (1996), Handbook on Bioethanol, Taylor & Francis, pp. 183-212
Non-Patent Document 6: Takashi Watanabe (2002), Biomass Handbook, Ohmsha, Ltd., pp. 176-183
Non-Patent Document 7: Munezo Takahashi (1989), Mushrooms and Wood (Kinoko to Mokuzai), Tsukiji Shokan
Non-Patent Document 8: Yuki Hashimoto et al. (2003), Abstracts of the 7th Annual Meeting of the Japanese Society of Mushroom Science and Biotechnology, the 7th Annual Meeting of the Japanese Society of Mushroom Science and Biotechnology, p. 67

### DISCLOSURE OF THE INVENTION

It is an objective of the present invention to develop a method of pretreating a waste mushroom bed so as to easily and efficiently obtain sugars and ethanol with the use of the waste mushroom bed. It is another objective of the present invention to develop a method of converting the pretreated waste mushroom bed to yield sugars and ethanol.

As a result of intensive studies to achieve the above objectives, the present inventors have found that it is possible to improve the efficiency of conversion of lignocellulose that remains in a waste mushroom bed to yield sugars and ethanol by enzyme treatment with the introduction of a step of treating wood by directly using mushroom hyphae in a waste mushroom bed. (Waste mushroom beds are obtained as waste products in large amounts after mushroom cultivation in factories and can be used for limited purposes.) This has led to the completion of the present invention.

Specifically, the present invention relates to a pretreatment method, wherein mushroom hyphae contained in a waste mushroom bed are directly used for treatment of lignocellulose, which is a component of wood in the waste mushroom bed, following which another pretreatment may optionally be carried out. Thereafter, ethanol is obtained by sequentially carrying out enzymatic saccharification and alcoholic fermentation with the use of a microorganism, or by carrying out multiple parallel fermentation, in which enzymatic saccharification and alcoholic fermentation with the use of a microorganism are simultaneously performed. Hereafter, the present invention will be described in detail.

The present invention relates to following (1) to (9):

(1) a method of pretreating a waste mushroom bed, comprising maintaining a waste mushroom bed at 4° C. to 30° C. for 1 week or longer after harvesting of fruit bodies for conversion of the waste mushroom bed to yield sugars and ethanol;

(2) the method of pretreating a waste mushroom bed according to (1) above, wherein the waste mushroom bed is maintained at 20° C. to 30° C.;

(3) the method of pretreating a waste mushroom bed according to (1) or (2) above, wherein the waste mushroom bed is maintained for 4 weeks or longer after harvesting of fruit bodies;

(4) the method of pretreating a waste mushroom bed according to any one of (1) to (3) above, wherein the waste mushroom bed is a waste mushroom bed used for edible mushrooms;

(5) the method of pretreating a waste mushroom bed according to (4) above, wherein the edible mushrooms are “maitake” (Grifola frondosa), “eryngii” (Pleurotus eryngii), “bunashimeji” (Hypsizigus marmoreus), “shiitake” (Lentinula edodes), or “nameko” (Pholiota nameko);

(6) a method of converting a waste mushroom bed to yield ethanol, comprising:

allowing a waste mushroom bed to be subjected to pretreatment according to any one of (1) to (3) above for conversion of the waste mushroom bed to yield sugars and ethanol; and

saccharifying the waste mushroom bed to yield glucose, xylose, mannose, arabinose and/or galactose with the use of an enzyme such as cellulase, hemicellulase, or cellulase mixed with hemicellulase followed by ethanol fermentation with the use of a microorganism; or

simultaneously carrying out saccharification with the use of the enzyme and ethanol fermentation with the use of a microorganism;

(7) a method of converting a waste mushroom bed to yield ethanol, comprising:

allowing a waste mushroom bed to be subjected to pretreatment according to any one of (1) to (3) above for conversion of the waste mushroom bed to yield sugars and ethanol;

allowing the waste mushroom bed to be subjected to alkaline treatment or pulverization treatment; and

saccharifying the waste mushroom bed to yield glucose, xylose, mannose, arabinose and/or galactose with the use of an enzyme such as cellulase, hemicellulase, or cellulase mixed with hemicellulase followed by ethanol fermentation with the use of a microorganism; or

simultaneously carrying out saccharification with the use of the enzyme and ethanol fermentation with the use of a microorganism;

(8) the method of converting a waste mushroom bed to yield ethanol according to (6) or (7) above, wherein the waste mushroom bed is a waste mushroom bed used for edible mushrooms; and

(9) the method of converting a waste mushroom bed to yield ethanol according to (8) above, wherein the edible mushrooms are “maitake,” “eryngii,” “bunashimeji,” “shiitake,” or “nameko.”

As a result of various studies, the present inventors have found that it is effective to treat lignocellulose, which is a component of wood contained in waste mushroom beds, with hyphae remaining in a waste mushroom bed for pretreatment for conversion of such lignocellulose to yield sugars such as glucose or ethanol. (The term “waste mushroom bed” used herein indicates a mushroom bed remaining after harvesting of fruit bodies of mushrooms such as “maitake,” “eryngii,” “bunashimeji,” “shiitake,” and “nameko,” which were subjected to bag or bottle cultivation in a medium containing sawdust powder). This has led to the completion of the present invention. The present invention will be described below in detail.

A method of pretreatment does not particularly require complex operations, and thus it is possible to achieve a desired objective by maintaining a waste mushroom bed at 4° C. to 30° C. for 1 week or longer after harvesting of fruit bodies. Preferably, a waste mushroom bed is maintained for 4 weeks or longer at 20° C. to 30° C., at which temperature mushrooms can readily grow. In such case, treatment effects can be further improved under conditions in which oxygen can be supplied to the waste mushroom bed. Further, it is preferable to prevent a waste mushroom bed from being excessively dried. After mushroom hyphae have spread in a waste mushroom bed, the waste mushroom bed is unlikely to become moldy while being maintained at an adequate temperature. Thus, it is not necessary to carry out large-scale sterilization. Furthermore, additional inoculation of fungi is unnecessary. Therefore, such waste mushroom bed is maintenance-free.

According to the method of such pretreatment, a waste mushroom bed in any form may be treated. Thus, during pretreatment, a waste mushroom bed may be placed in a bag or bottle, or may be in the form of, for example, a field heap.

For instance, in the case of maitake fungi, significant effects can be obtained by treatment at an optimum temperature for fungal growth of approximately 25° C. with sufficient ventilation. After approximately 12 weeks of such treatment, the efficiency of conversion into sugars such as glucose or ethanol with the use of an enzyme is 3.5 to 10 times greater than that before treatment.

During such treatment, due to transition from a generative growth phase to a vegetative growth phase, mushroom hyphae contained in a waste mushroom bed resume secretion of an enzyme such as a lignin-degrading enzyme, which is represented by laccase, after suspension of secretion of the enzyme. It is considered that such enzyme or hyphae would directly act on lignocellulose, which is a component of wood in a waste mushroom bed, such that pretreatment of the waste mushroom bed is carried out.

After such pretreatment, it is also possible to improve the rate of conversion into sugars such as glucose or ethanol by carrying out some sort of treatment. In such case, a treatment following pretreatment may be any form of treatment that is known as a lignocellulose biomass pretreatment, such as a physical or a chemical treatment. In the case of a waste mushroom bed, alkaline treatment or pulverization treatment is more effective.

In the case of alkaline treatment, a waste mushroom bed may be heat-treated at 100° C. in a 1% to 5% NaOH solution. In the case of pulverization treatment, it is further effective to use an oscillating pulverizer in a manner such that 70% or more of pulverized particles have particle sizes of not more than 90 μm.

After such treatment, sugars such as glucose are generated from a waste mushroom bed by enzymatic saccharification, following which such sugars are subjected to ethanol fermentation with the use of a microorganism. Alternatively, conversion into ethanol is carried out by multiple parallel fermentation, in which enzymatic saccharification and ethanol fermentation with the use of a microorganism are simultaneously performed.

In the above case, it is possible to obtain sugars such as glucose by enzymatic saccharification alone without ethanol fermentation with the use of a microorganism. In a case in which the enzyme used is cellulase, glucose derived from cellulose contained in lignocellulose can be obtained. In a case in which hemicellulase such as xylanase is used, sugars such as xylose, mannose, arabinose, and galactose, which are derived from hemicellulose contained in lignocellulose, can be obtained. In addition, with the use of cellulase mixed with hemicellulase, it is possible to simultaneously obtain sugars such as glucose derived from cellulose and xylose derived from hemicellulose. It is also possible to convert the thus obtained sugars into non-ethanol substances.

In particular, upon the use of a treated waste mushroom bed according to the present invention, culture is carried out using yeast and an enzyme such as cellulase, hemicellulase, or cellulase mixed with hemicellulase. Thus, enzymatic activity inhibition caused by sugars generated by saccharification is less likely to occur during multiple parallel fermentation, in which saccharification and ethanol fermentation (of sugars generated by saccharification) with the use of a microorganism such as yeast are simultaneously performed, resulting in more effective fermentation progression.

An enzyme used for fermentation may be a commercially available product. Also, a culture solution containing cultured filamentous fungi or a purified product of such culture solution may be used as long as cellulose or hemicellulose can be saccharified. Commercially available enzymes or partially purified enzymes often contain cellulase mixed with hemicellulase. The amount of enzyme may be adequately determined. In the case of a maitake waste mushroom bed, it is effective to add cellulase mixed with hemicellulase at 12.5-50 FPU (filter paper units (filter paper degradation activity)) per mushroom bed. In the case of an enzyme in a powder form, such enzyme is suspended in a buffer at approximately pH 5.0 for convenient use. Contamination of a fermentation system due to the presence of germs can be prevented by first removing germs from an enzyme solution with the use of a filter that is 0.45 μm or less in size.

It is convenient and effective to use “Saccharomyces cereviciae” yeast as a microorganism used for ethanol fermentation. Meanwhile, in a case in which pentose such as xylose derived from hemicellulose is subjected to ethanol fermentation, Pichia stipitis can be used. Also, salt-tolerant Shizosaccharomyces pombe or the like can be used depending on conditions. Further, in the case of the use of a non-yeast organism, an organism such as Zymomonas mobilis, which is a bacterium that can cause ethanol fermentation, and a gene recombinant thereof may be used as long as ethanol fermentation can be carried out. In the case of the use of S. cereviciae, S. cereviciae preserved in a slant medium or cryopreserved S. cereviciae may be used. Alternatively, commercially available bakers' yeast may be used. Bakers' yeast in dried or raw form is directly introduced into a fermentation system such that fermentation is efficiently carried out due to the presence of the yeast at a high concentration following the commencement of fermentation. When a yeast that has been preserved in a slant medium is used, such yeast is precultured with the use of a liquid medium before multiple parallel fermentation. This is because it is desirable to increase the amount and activity of such yeast.

Any medium may be used as a liquid medium for preculture as long as such medium is appropriate for yeast culture. For instance, such medium may be 1% yeast extract, 2% peptone, and 3% glucose and have a pH of 5.0. After the termination of preculture, yeast cells are recovered and used. Fermentation can be carried out without difficulty with the addition of yeast at a final concentration of 0.1 g/l or more. With the use of yeast in large amounts, good fermentation efficiency is achieved, as described above, and it is possible to prevent contamination.

The content of aforementioned treated waste mushroom bed may be added in an adequate amount relative to the amount of fermentation product. However, with the use of waste mushroom bed content at a high concentration, it becomes difficult to perform agitation at the beginning of fermentation due to high viscosity. Therefore, it is preferable to adjust the amount of such treated waste mushroom bed content to be introduced to a level at which sufficient agitation can be carried out based on consideration of mixer performance.

A fermentation solution to which the treated waste mushroom bed and nutrient sources necessary for yeast growth have been added is sterilized in an autoclave (121° C., 15 minutes or more). After sterilization, the fermentation solution is cooled to approximately 37° C. Then, the aforementioned enzyme or yeast is introduced thereinto, and fermentation is initiated at 37° C. During fermentation, fermentation efficiency can be improved by agitation under anaerobic conditions. As above, culture is carried out for 1 to 3 days such that cellulose and hemicellulose contained in the waste mushroom bed can be converted into ethanol.

### EFFECTS OF THE INVENTION

According to the present invention, with the reuse of mushroom hyphae remaining in a waste mushroom bed after mushroom cultivation, it is possible to obtain sugars and ethanol at high yields from lignocellulose contained in such waste mushroom bed under moderate production conditions without exceptional operations.

This description includes part or all of the contents as disclosed in the description of Japanese Patent Application No. 2005-053831, which is a priority document of the present application.

### BEST MODE FOR CARRYING OUT THE INVENTION

The present invention is hereafter described in greater detail with reference to the following examples, although the technical scope of the present invention is not limited thereto.

### EXAMPLES

### (1) Storage Treatment of Maitake Waste Mushroom Bed

Beech sawdust powder and corn bran were mixed at a volume ratio of 9:1. The moisture content of the mixture was adjusted to 65%. Accordingly, a maitake cultivation medium was produced using such mixture. The content (excluding the water content) of beech sawdust powder and corn bran were 80% and 20% by weight, respectively. The medium was placed in a maitake cultivation bag (2.5 kg in capacity), followed by sterilization for 105° C. for 2 hours. After cooling, the medium was inoculated with maitake fungi, followed by culture at approximately 25° C. for 2.5 months. Then, the cultivation bag was transferred to a room at a temperature of approximately 16° C. and the upper part of the cultivation bag was cut off for maitake fruit body development. At the optimum time for harvesting fruit bodies, the fruit bodies were harvested. Accordingly, a waste mushroom bed was obtained.

The obtained waste mushroom bed was taken out from the maitake cultivation bag and placed in another maitake cultivation bag. In order to secure oxygen supply, the part above the filter part of the cultivation bag was sealed with a sealer. In addition, treatment was carried out at different test temperatures under conditions in which gas exchange between the inside and the outside of the bag was allowed to take place.

The treatment period was arbitrarily determined to be between 1 to 12 weeks. The waste mushroom bed was taken out after the termination of the treatment period. The waste mushroom bed in a block shape was flaked, sufficiently agitated, and then used for subsequent treatment.

### (2) Alkaline Treatment of the Pretreated Waste Mushroom Bed

The above pretreated waste mushroom bed was placed in a plastic bottle in an amount of 20 g in terms of dry weight. A 5% NaOH solution (100 ml) was poured thereinto. After sufficient mixing, the plastic bottle was covered by a wrap in a manner such that the whole waste mushroom bed was able to soak in the NaOH solution, followed by autoclaving at 100° C. for 60 minutes. After the termination of autoclaving, the waste-mushroom-bed-containing NaOH solution was cooled down to room temperature. The pH of the waste-mushroom-bed-containing NaOH solution was 12.5. Therefore, the pH was lowered to approximately 7.0 with the use of sulfuric acid. Thereafter, the alkaline-treated waste mushroom bed was washed with running water with the use of an 80-mesh sieve until washing waste water became transparent. The alkaline-treated waste mushroom bed subjected to washing was dried using a dryer and then used for a saccharification treatment or multiple parallel fermentation.

### (3) Pulverization Treatment of the Pretreated Waste Mushroom Bed

The above pretreated waste mushroom bed was dried overnight at 60° C. in a drying oven. The thus dried maitake waste mushroom bed (approximately 200 g) was placed in a polyethylene case (4.5 liters in capacity). Further, 1.2 kg of zirconia balls each 10 mm in diameter were added thereto as media. The cover of the case was tightly closed and the case was placed in an oscillating mill. The oscillation frequency was set at 50.0 Hz and pulverization was then initiated. The pulverization time was predetermined to be 2 hours. However, in order to avoid heat generation, the mill was operated for 2 hours in total in the following manner: the mill was operated for 30 minutes; the operation was terminated; the case and the sample were cooled down; and the mill was operated for another 30 minutes. After the termination of pulverization, the mixture of pulverized particles of the waste mushroom bed and the media was placed on a 4.0 mm-mesh sieve, followed by sieving. Thus, the pulverized particles of the waste mushroom bed were separated from the media.

### (4) Enzymatic Saccharification of the Maitake Waste Mushroom Bed

An enzyme solution comprising a 50 mM citrate buffer to which 60 FPU/g-biomass cellulase (mixed with hemicellulase) and 1 mM sodium azide had been added was prepared. The enzyme solution was dispensed in 10 ml portions into 50-ml Erlenmeyer flasks, each of which contained 100 mg of the treated maitake waste mushroom bed obtained in (1) to (3) above. The flasks were covered to avoid moisture evaporation, followed by a reaction at 50° C. for 3 days with shaking at 120 rpm. After the termination of the reaction, sampling of the solution in a required amount was carried out. Each solution was maintained in boiling water for 5 minutes for enzyme deactivation. The insoluble matter was removed therefrom by centrifugation. Thereafter, glucose concentration in each solution was measured using a glucose sensor. At such time, the xylose concentration was measured using a xylose sensor in combination therewith.

A saccharification rate was represented as the percentage of the amount of glucose actually generated from cellulose to the ideal glucose amount, provided that the cellulose content in a waste mushroom bed is 45%. In the cases in which alkaline treatment was carried out, the saccharification rate was calculated as the yield obtained from a waste mushroom bed prior to alkaline treatment with the consideration of weight loss due to alkaline treatment.

### (5) Conversion of the Maitake Waste Mushroom Bed to Yield Ethanol

### Multiple Parallel Fermentation

Ethanol conversion was carried out in a system containing 40 ml of a fermentation solution. Specifically, 30 ml of a 50 mM citrate-phosphate buffer (pH 5.0) was placed in a 100-ml Erlenmeyer flask. A treated waste mushroom bed (4.0 g (final concentration: 10%)) was mixed therewith. The pH of the resulting solution was adjusted to 5.0 with the use of concentrated phosphoric acid. The resulting solution was autoclaved at 121° C. for 15 minutes, followed by cooling to room temperature. Dry yeasts were added to a sterilized 50 mM citrate-phosphate buffer (pH 5.0) to a dry yeast concentration of 10 g/l, followed by sufficient agitation. Thus, a yeast solution was obtained. In addition, cellulase powder (30 FPU) was suspended in 2 ml of a buffer such that a cellulase solution was obtained. The cellulase solution was subjected to filter sterilization using a 0.42-μm filter.

The yeast solution (4 ml) and the cellulase solution (4 ml) were aseptically added to 32 ml of the waste mushroom bed solution in a clean bench such that 40 ml of the mixture solution was obtained. The Erlenmeyer flask was closed with a fermentation plug that had been sterilized with ethanol and the gap around the plug was covered with Parafilm. The thus prepared flask was placed in an incubator set at 37° C., followed by fermentation (culture) for 7 days with shaking (120 rounds). After the termination of fermentation, the supernatant and the solid content were separated from each other by centrifugal separation. Then, the ethanol concentration of the supernatant was measured by gas chromatography.

Table 1 shows results of saccharification or ethanol conversion of a maitake waste mushroom bed subjected to treatment at 25° C. for 1, 4, or 12 week(s). Table 2 shows results of examining the influence of treatment temperatures. In table 2, the saccharification rates at different temperatures are shown as values relative to the saccharification rate of an untreated waste mushroom bed, which is designated as 100. Further, table 2 shows results obtained with and without alkaline treatment. Table 3 shows results of saccharification or ethanol conversion, prior to which alkaline treatment was carried out after treatment similar to that in table 1. Table 4 shows results of saccharification, prior to which pulverization treatment was carried out after treatment similar to that in table 1.

TABLE 1
Influence of maitake waste mushroom bed treatment time
upon saccharification rate and ethanol conversion rate

| | | Saccharification | Ethanol |
|---|---|---|---|
| | Treatment | rate | conversion rate |
| Test group | week | (%) | (%) |
| Untreated | 0 | 5.8 | 9.5 |
| (control) | | | |
| 1-week treatment | 1 | 10.2 | — |
| 4-week treatment | 4 | 29.0 | 32.5 |
| 12-week | 12 | 50.9 | 42.2 |
| treatment | | | |

TABLE 2
Influence of maitake waste mushroom bed treatment
temperature upon saccharification rate

| | | Relative value of | Relative value of |
|---|---|---|---|
| | | saccharification | saccharification |
| Treatment | Treatment | rate (without | rate (with alkaline |
| temperature | week | alkaline treatment) | treatment) |
| Untreated | 0 | 100 | 100 |
| (control) | | | |
| 4° C. | 4 | 121.1 | 111.9 |
| 25° C. | 4 | 500.0 | 152.9 |
| 29° C. | 4 | 230.3 | 115.8 |
| 33° C. | 4 | 61.0 | 95.6 |
| 37° C. | 4 | 101.1 | 40.9 |

TABLE 3
Influence of maitake waste mushroom bed treatment
time upon saccharification rate and ethanol conversion
rate (in combination with alkaline treatment)

| | | Saccharification | Ethanol |
|---|---|---|---|
| | Treatment | rate | conversion rate |
| Test group | week | (%) | (%) |
| No treatment + alkaline | 0 | 41.4 | 34.2 |
| treatment | | | |
| 1-week treatment + | 1 | 51.0 | — |
| alkaline treatment | | | |
| 4-week treatment + | 4 | 63.3 | 42.2 |
| alkaline treatment | | | |
| 12-week treatment + | 12 | 54.2 | 40.2 |
| alkaline treatment | | | |

TABLE 4
Influence of maitake waste mushroom bed treatment time
upon saccharification rate and ethanol conversion rate
(in combination with pulverization treatment)

| | | Saccharification |
|---|---|---|
| | Treatment | rate |
| Test group | week | (%) |
| No treatment + pulverization | 0 | 32.2 |
| treatment | | |
| 4-week treatment + pulverization | 4 | 50.9 |
| treatment | | |
| 12-week treatment + pulverization | 12 | 54.2 |
| treatment | | |

TABLE 5
Influence of maitake waste mushroom
bed treatment time upon xylose yield

| | | Xylose |
|---|---|---|
| | Treatment | concentration* |
| Test group | week | (g/L) |
| No treatment (control) | 0 | 0.2 |
| 4-week treatment | 4 | 0.3 |
| 4-week treatment + alkaline treatment | 4 | 1.7 |
| 12-week treatment | 12 | 0.7 |
| 12-week treatment + alkaline treatment | 12 | 1.4 |

*Increased xylose concentration in a reaction solution subjected to enzyme treatment

As is apparent from table 1, in the case of a waste mushroom bed treated at 25° C., the saccharification rate was found to have approximately doubled over a 1 week treatment time. The longer the treatment time, the greater the saccharification rate. Similar results were obtained regarding the ethanol conversion rate. Further, as is apparent from table 2, treatment effects were observed between 4° C. and approximately 30° C. As is apparent from table 3 or 4, with alkaline treatment or a pulverization treatment following the above treatment, the saccharification rate further increases. For instance, when alkaline treatment or a pulverization treatment was not carried out during 4-week treatment, the saccharification rate was 29.0% (table 1). However, when alkaline treatment was carried out, the saccharification rate was 63.3% (table 3). Also, when pulverization treatment was carried out, the saccharification rate was 50.9% (table 4). In each case, the saccharification rate obtained was twice as great as that without alkaline treatment or pulverization treatment. In such cases, it is understood that significant effects can be obtained with treatment for 4 weeks or longer.

Table 5 lists xylose yields. It is understood that, as with the case of glucose, the xylose yield can be increased 3.5 times and 8.5 times over a 12-week treatment and 4-week treatment followed by an alkaline treatment, respectively.

### (6) Storage Treatment of Non-Maitake Waste Mushroom Bed

Storage treatment was carried out using a non-maitake waste mushroom bed obtained after bottle cultivation of bunashimeji or eryngii. In the case of bunashimeji, a medium comprising hardwood sawdust powder was adjusted to have a moisture content of 65%. The medium (approximately 630 g) was placed in a 850-cc bottle, followed by sterilization, cooling, and inoculation with bunashimeji fungi. Culture was carried out at 25° C., followed by fruit body development at 14° C. At the optimum time of harvesting of the fruit bodies, harvesting was carried out. Then, the medium left in the bottle was used as a waste mushroom bed.

In the case of eryngii, a medium comprising conifer sawdust powder was adjusted to have a moisture content of 71%. The medium (approximately 580 g) was placed in a 850-cc bottle, followed by sterilization, cooling, and inoculation with eryngii fungi. Culture was carried out at 23° C., followed by fruit body development at 17° C. At the optimum time for harvesting the fruit bodies, harvesting was carried out. Then, the medium left in the bottle was used as a waste mushroom bed.

The bottle containing the thus obtained waste mushroom bed was placed in a maitake cultivation bag. The part of the bag above the filter part was sealed by pressure bonding in a manner such that gas exchange was achieved. The bag was maintained at 25° C. for 4 weeks. After the termination of the storage period, the above waste mushroom bed was taken from the bottle and was sufficiently agitated and dehydrated. Thereafter, alkaline treatment and cellulose saccharification were carried out in the manner described above.

TABLE 6
Results of saccharification of waste mushroom bed subjected
to storage treatment using mushroom fungi (excluding
maitake fungi) (with alkaline treatment)

| | | Glucose concentration | | |
|---|---|---|---|---|
| | | (Relative value: 100 in week 0) | | |
| Mushroom | Temperature | Week 0 | Week 4 | Week 12 |
| Bunashimeji | 25° C. | 100 | 117 | 123 |
| Eryngii | 25° C. | 100 | 116 | 153 |

As is apparent from table 6, it is possible to improve saccharification rate in the cases of bunashimeji and eryngii with alkaline treatment following treatment at 25° C.

In addition, it has been confirmed that similar effects can be obtained also in the cases of shiitake and nameko.

All publications, patents, and patent applications cited herein are incorporated herein by reference in their entirety.

1. A method of pretreating a waste mushroom bed, comprising maintaining a waste mushroom bed at 4° C. to 30° C. for 1 week or longer after harvesting of fruit bodies for conversion of the waste mushroom bed to yield sugars and ethanol.

2. The method of pretreating a waste mushroom bed according to claim 1, wherein the waste mushroom bed is maintained at 20° C. to 30° C.

3. The method of pretreating a waste mushroom bed according to claim 1, wherein the waste mushroom bed is maintained for 4 weeks or longer after harvesting of fruit bodies.

4. The method of pretreating a waste mushroom bed according to claim 1, wherein the waste mushroom bed is a waste mushroom bed used for edible mushrooms.

5. The method of pretreating a waste mushroom bed according to claim 4, wherein the edible mushrooms are “maitake” (Grifola frondosa), “eryngii” (Pleurotus eryngii), “bunashimeji” (Hypsizigus marmoreus), “shiitake” (Lentinula edodes), or “nameko” (Pholiota nameko).

6. A method of converting a waste mushroom bed to yield ethanol, comprising: allowing a waste mushroom bed to be subjected to pretreatment according to any one of claims 1 to 3 for conversion of the waste mushroom bed to yield sugars and ethanol; and saccharifying the waste mushroom bed to yield glucose, xylose, mannose, arabinose and/or galactose with the use of an enzyme such as cellulase, hemicellulase, or cellulase mixed with hemicellulase followed by ethanol fermentation with the use of a microorganism; or simultaneously carrying out saccharification with the use of the enzyme and ethanol fermentation with the use of a microorganism.

7. A method of converting a waste mushroom bed to yield ethanol, comprising:
allowing a waste mushroom bed to be subjected to pretreatment according to any one of claims 1 to 3 for conversion of the waste mushroom bed to yield sugars and ethanol;
allowing the waste mushroom bed to be subjected to alkaline treatment or pulverization treatment; and
saccharifying the waste mushroom bed to yield glucose, xylose, mannose, arabinose and/or galactose with the use of an enzyme such as cellulase, hemicellulase, or cellulase mixed with hemicellulase followed by ethanol fermentation with the use of a microorganism; or
simultaneously carrying out saccharification with the use of the enzyme and ethanol fermentation with the use of a microorganism.

8. The method of converting a waste mushroom bed to yield ethanol according to claim 6, wherein the waste mushroom bed is a waste mushroom bed used for edible mushrooms.

9. The method of converting a waste mushroom bed to yield ethanol according to claim 8, wherein the edible mushrooms are “maitake,” “eryngii,” “bunashimeji,” “shiitake,” or “nameko.”

10. The method of pretreating a waste mushroom bed according to claim 2, wherein the waste mushroom bed is maintained for 4 weeks or longer after harvesting of fruit bodies.

11. The method of pretreating a waste mushroom bed according to claim 2, wherein the waste mushroom bed is a waste mushroom bed used for edible mushrooms.

12. The method of pretreating a waste mushroom bed according to claim 11, wherein the edible mushrooms are “maitake” (Grifola frondosa), “eryngii” (Pleurotus eryngii), “bunashimeji” (Hypsizigus marmoreus), “shiitake” (Lentinula edodes), or “nameko” (Pholiota nameko).

13. The method of pretreating a waste mushroom bed according to claim 3, wherein the waste mushroom bed is a waste mushroom bed used for edible mushrooms.

14. The method of pretreating a waste mushroom bed according to claim 13, wherein the edible mushrooms are “maitake” (Grifola frondosa), “eryngii” (Pleurotus eryngii), “bunashimeji” (Hypsizigus marmoreus), “shiitake” (Lentinula edodes), or “nameko” (Pholiota nameko).

15. The method of converting a waste mushroom bed to yield ethanol according to claim 7, wherein the waste mushroom bed is a waste mushroom bed used for edible mushrooms.

16. The method of converting a waste mushroom bed to yield ethanol according to claim 15, wherein the edible mushrooms are “maitake,” “eryngii,” “bunashimeji,” “shiitake,” or “nameko.”
